(19) United States
(12) Patent Application Publication
(10) Pub. No.: US 2019/0056389 A1
(43) Pub. Date: Feb. 21, 2019

(54) System and method for determining the presence or absence of adsorbed biomolecules or biomolecular structures on a surface

(71) Applicants: Aykutlu Dana, Stanford, CA (US); Sencer Ayas, Sunnyvale, CA (US); Ayse Begum Tekinay, Ankara (TR)

(72) Inventors: Aykutlu Dana, Stanford, CA (US); Sencer Ayas, Sunnyvale, CA (US); Ayse Begum Tekinay, Ankara (TR)

(21) Appl. No.: 15/998,396

(22) Filed: Aug. 15, 2018

Related U.S. Application Data
(60) Provisional application No. 62/545,897, filed on Aug. 15, 2017.

(51) Int. Cl. G01N 33/543

(52) U.S. Cl. CPC G01N 33/54373; G01N 33/54393

(57) ABSTRACT

An optical sensing method with optical excitation in the ultraviolet (UV) range (200-400 nm wavelength) where biomolecules, which have inherent absorption lines in the UV range, adsorb on a plasmonic surface exhibiting coincident optical resonances in the UV, and the amplitude of the reflected/scattered/transmitted optical wave, which is strongly changed due to the presence of biomacromolecules or biomolecular structures, are measured or imaged to infer the presence and optical properties of the adsorbed biomolecular structures.

The current application claims a priority to the U.S. Provisional Patent application Ser. No. 62/545,897 filed on Aug. 15, 2017.

### FIELD OF THE INVENTION

The present invention is in the technical field of biomolecular sensing. More particularly, the present invention is in the technical field of plasmon resonance-based sensing of biomacromolecules. In addition, the present invention is in the technical field of photonics. More particularly, the present invention is in the technical field of plasmon resonance-based sensing. More particularly, the present invention is in the technical field of multiply-resonant plasmon resonant sensing and imaging.

### BACKGROUND OF THE INVENTION

Optical techniques that identify and measure biomacromolecules such as proteins, glycosaminoglycans and nucleic acids frequently require fluorescent or enzymatic labels as well as a means of isolating or separating analytes. Label-free techniques such as surface plasmon resonance (SPR) separate analytes in complex mixtures through the use of specific capture ligands, usually antibodies, bonded to a metallic surface in contact with a dielectric, and measure the presence of bound biomolecules by optically measuring the reflectance/scattering/transmittance off the metallic surface. The sensitivity of present SPR systems with wavelengths in the visible or near-infrared portions of the spectrum is insufficient to observe small molecules and biomolecular complexes at low enough concentrations or surface coverage. The present invention aims to improve the sensitivity of label-free plasmon resonance-based sensors for the detection of biomolecules, their complexes, or larger biological entities such as viruses, bacteria or exosomes.

Light at a specific wavelength striking the metal/dielectric interface at a specific angle can support a rapidly decaying wave phenomenon (surface plasmon) if there is a means of matching the momentum (K-vector) of the light with that of the loosely bound electrons at the metal/dielectric interface. When this resonance energy transfer occurs, the intensity of the light reflected from the metal surface decreases markedly. This resonance phenomenon is quite sharp (on the order of a few millidegrees to few degrees depending on excitation conditions and grating shape), and the incident angle is extremely sensitive to the refractive index at the surface of the metal substrate. In the present invention, a diffraction grating is embossed on a thick (100 nm) aluminum layer with two additional layers on top of it or a thin aluminum layer with the additional layers on an underlying grating. The diffraction grating also provides the momentum matching mechanism at ultraviolet (UV) wavelengths where the biomolecules have enhanced refractive indices and light absorption, resulting in an enhanced sensitivity for the overall detection process.

Typically, antibodies bound to the metal surface are used to capture specific analytes present in a complex sample mixture which shows over the metal surface. This highly specific immunochemical process results in binding of the specific analytes to well-identified regions of the metal substrate without the necessity of physical compartmentalization of the fluid. For each captured analyte, the magnitude of the change in the resonant angle is proportional to the mass of analyte captured in each region. With appropriately designed accommodations, an SPR analyzer can be made to capture living eukaryotic cells (or viruses or bacteria) by binding to surface antigens normally expressed on the surface of the cells. In this manner, specific cell types, distinguished by their surface antigens can be isolated and captured on a metal surface. Cells captured in an SPR analyzer in this manner can be activated by contact with suitable ligands for distinct cell surface receptors.

Captured antibodies for various cell secretions (cytokines) can be spotted on the surface in order to immobilize the secreted cytokines. Cytokines are relatively small molecules and the amount of a particular cytokine secreted by a single cell is typically too small to be detected by SPR resonance angle shifts. Also, conventional SPR systems do not possess enough sensitivity to detect certain molecules at concentrations that are encountered in healthy blood serum or plasma. Therefore, despite the wide availability of the technique, improvement of sensitivity for the detection of biomolecules and biological structures is highly desirable in plasmon resonance-based sensing.

A quick survey of the literature points to the success of surface plasmon resonance (SPR) biosensors in a wide range of fields from fundamental biological studies to clinical diagnosis applications (Rich and Myszka, 2005; Shankaran et al., 2007). While much of this success has been recent, SPR has long played a role in surface sciences. In the early 20th century, the excitation of surface plasmons, initially termed Wood's Anomalies, was observed as anomalous reflective patterns when polarized light was shown on a metal grating (Wood, 1902). The phenomenon was attributed to the resonant coupling of photons from the polarized light to the oscillation of metal-free electrons (surface plasmon polaritons), generating in the process a strong electromagnetic evanescent wave bound to the metal surface. This phenomenon was widely applied to the study of thin metal films and coatings. Much of the pioneering work in describing the unique properties of surface plasmons, the methods for its resonant excitation and its use in sensing has been carried out by Ritchie et al. (1968), Raether (1988), and Nylander et al. (1982). Today, biosensors using the excitation of the surface plasmons are generally termed surface plasmon resonance (SPR). Those biosensors provide rich information on the specificity, affinity, and kinetics of biomolecular interactions and/or the concentration levels of an analyte of interest from a complex sample (Shankaran et al., 2007). The analysis is done in real-time and without requiring fluorophore labeling. Currently, numerous commercial systems are available; however, their designs do not differ significantly from the original concept described by Liedberg et al. (1983) to demonstrate SPR biosensing. The literature contains numerous examples of novel SPR biosensor designs that improve upon the traditional and popular prism-coupled SPR (Kretchmann's configuration). Homola's 1999 review paper and his follow-up article in 2003 provide an excellent overview of the development from the past 20 years (Homola, 2003; Homola et al., 1999). Recently, much of the development of SPR is directed towards providing an integrated, low-cost, reusable and sensitive biosensor.

In SPR biosensing, the adsorption of a targeted analyte by a surface bioreceptor is measured by tracking the change in the conditions of the resonance coupling of incident light to the propagating surface plasmon wave (SPW). The existence of this surface plasmon wave is dictated by the electromagnetic (EM) properties of a metal, typically gold or silver, and a dielectric (sample-medium) interface. The resonance coupling appears as a dip in the reflectivity of the light spectrum, which is traditionally tracked by measuring the wavelength, the incident angle, or the intensity of the reflected light. The coupling of the light to the SPW requires, for electromagnetic reasons, a high-index prism or a periodic grating surface (Raether, 1988). The sensitivity of the SPR lies in the strong electromagnetic enhancement of the SPW. Commercial SPR biosensors are generally capable of detecting 1 pg/mm² of absorbed analytes. This sensitivity is strongly dependent on many parameters, but is particularly dependent on surface functionalization. In comparing sensitivity between the reported SPR biosensors, one must be cautious as the sensitivity values are often described independently of the surface functionalization chemistry or for a specific application. For a more relevant assessment, the sensitivity, where it is available, is typically expressed in terms of detectable refractive index unit (RIU) change. This value strictly reflects the performance of the optical configuration, the measurement approach or the data analysis algorithm. Commercial systems are reported with sensitivity typically within 1×10⁻⁵ to 1×10⁻⁶ RIU. However, the RIU sensitivity is not a direct indication of the lowest concentration of biomolecules that can be detected with an SPR system, since sensitivity to bulk refractive index changes can be significantly different than sensitivity to adsorbed biomolecular layers. Today, the key challenge in the SPR biosensor development lies not primarily in the integration of the various components of the biosensor (sampling handling, control electronics, etc.) but on providing robust integrated SPR biosensors that are as or more sensitive (<pg/mm²) than their current counterparts. Beyond the requirement for high-sensitivity, in particular for the detection of small biomolecules, low cost of production, compact design, reusability and increased functionality (multiple analyte detection, temperature control, etc.) are as well sought.

The surface plasmon resonance is significantly affected by a thin analyte layer on the metal layer and as well as by the bulk refractive index changes. In the absence of an analyte layer the SPR angle (θ_{SPR}) is determined by:

$${θ}_{SPR} = {sin}^{- 1} ℝ e [ \frac{{k}_{SP}}{{k}_{o} {n}_{p}} ] \qquad ( Eq . 2 )$$

where k₀=2π/λ is the free space wavevector magnitude, and k_{SP} is the surface plasmon polariton (SPP) wavevector magnitude given as:

$${k}_{SP} = {k}_{0} \sqrt{\frac{{ϵ}_{d} {ϵ}_{m}}{{ϵ}_{d} + {ϵ}_{m}}} \qquad ( Eq . 3 )$$

where ∈_{d} and ∈ₘ are the complex dielectric constants of the bulk and the metal supporting the SPP, respectively. The change in the SPP wavevector upon deposition of an analyte layer of thickness h is given by:

$$Δ {k}_{SP} ≅ ℝ e [ \frac{{k}_{SP}^{3}}{{k}_{o}^{2} {n}_{d}^{3}} Δ {n}_{org} ( 1 - exp ( - 2 {γ}_{d} h ) ) ] \qquad ( Eq . 4 )$$

where Δn_{org}=n_{org}−n_{d} is the refractive index contrast between the organic layer index and the bulk (analyte buffer) and γ_{d}=ik₀∈_{d}/\sqrt{∈_{d}+∈ₘ}. The change in the SPR angle upon changes in the bulk refractive index (Δn_{d}) and the analyte layer thickness of h is then approximated as:

$${Δθ}_{SPR} = \frac{1}{{k}_{o} {n}_{p} cos {θ}_{SPR}} ℝ e [ \frac{{k}_{SP}^{3}}{{k}_{o}^{2} {n}_{d}^{3}} Δ {n}_{d} + \frac{2 {γ}_{d} {k}_{SP}^{3}}{{k}_{o}^{3} {n}_{d}^{3}} Δ {n}_{org} h ] \qquad ( Eq . 5 )$$

The approximation remains valid for h<<1/˜233 nm for a wavelength of 700 nm on Au/water interface. Gold and Silver cannot be used effectively in the UV range for plasmonic sensing, as their optical properties do not allow high quality factor plasmonic resonances due to intrinsic material losses. When aluminum is used as the metal and wavelengths are chosen in the UV part of the spectrum (200-400 nm), the field penetration depth into the dielectric decreases to several tens of nanometers, enhancing the sensitivity, which is a subject of this invention. However, adsorption to aluminum is less effective compared to gold, and gold is optically lossy in the UV range. Therefore, the optimal thickness of gold must be used not to over-dampen the plasmonic resonance on the aluminum-dielectric interface. In the present invention, a thin barrier layer of Al₂O₃ or SiO₂ is used on the aluminum surface as a protection layer to the adverse chemical effects of the biological buffer solutions on the aluminum, without significantly degrading the quality factors of the plasmonic resonances. Equation 5 captures the shift in θ_{SPR} with accumulation of an analyte layer or varying bulk refractive index close to the actual values calculated using the transfer matrix method (TMM) especially for the longer wavelengths resulting in larger penetration depths (1/γ_{d}).

The grating coupled configuration can offer a simpler setup by eliminating the need for a prism. When illuminated by a collimated beam at a specific angle, the grating structure exhibits resonances whose wavelength and quality factor depends on the grating period and groove depth. The patterning requirement can be eased when large-area patterning techniques are used for the fabrication or low-cost commercial grating surfaces. The SPR resonance for the grating coupled configuration can be observed as a minimum in the reflectance intensity when excited by transverse electric (TE) or transverse magnetic (TM) polarization, depending on the orientation of the grating with respect to the plane of incidence of the excitation beam. The sensitivity of the grating coupled SPR configuration to the bulk and the analyte layer can be expressed as:

$${Δθ}_{GCSPR} = \frac{1}{{k}_{o} {n}_{d} cos {θ}_{GCSPR}} ℝ e [ \frac{{k}_{SP}^{3}}{{k}_{o}^{2} {n}_{d}^{3}} Δ {n}_{d} + \frac{2 {γ}_{d} {k}_{SP}^{3}}{{k}_{o}^{3} {n}_{d}^{3}} Δ {n}_{org} h ] \qquad ( Eq . 6 )$$

where the resonant coupling angle (θ_{OCSPR}) is calculated through:

$${θ}_{GCSPR} = {sin}^{- 1} ℝ e [ \frac{{k}_{SP} - \frac{2 π m}{{Λ}_{g}}}{{k}_{0} {n}_{d}} ] \qquad ( Eq . 7 )$$

where m is an integer defining the order and Λ_{G} is the grating period.

For conventional SPR systems working in the visible or near infrared part of the spectrum (400 nm-1800 nm), the SPR angle or wavelength shift upon adsorption is dependent on the optical thickness Δn_{org}^{h} of the adsorbed species, and refractive indices of biomolecules and biomolecular structures are typically near n=1.47, and without strong absorption.

### SUMMARY OF THE INVENTION

The present invention is an optical method for examining the adsorption of biomacromolecules, including proteins and nucleic acids and small biologically relevant molecules such as drug molecules, or larger biological entities such as viruses, exosomes and bacteria, on surfaces.

This method comprises utilizing ultraviolet wavelengths (200-350 nm) for the excitation of surface plasmons or localized plasmons on appropriately designed photonic surfaces with resonances in the ultraviolet region, where biomolecules and other stated biologically relevant structures have inherent characteristic absorption lines based on their composition; and recording the changes in the intensity of reflected/scattered/transmitted light from the plasmonic surface in the ultraviolet region, achieving improved sensitivity to the adsorption of biomolecules and gross biological entities such as viruses, exosomes and microorganisms. The method also comprises determining the optical thickness and absorption coefficient of the thin adsorbed biomolecular layers or individual molecules through analysis of intensity of the angular or wavelength dependence of the reflected light. The method also comprises imaging the presence of biomacromolecules, viruses or other biological nanostructures under the condition of multiple-resonance, by choosing the excitation wavelength, the plasmonic resonance wavelength and the molecular absorption wavelengths to coincide, thereby improving sensitivity.

The present invention is a method for enhanced plasmonic sensing and imaging, by choosing the excitation wavelength to coincide with the native molecular absorption of biomolecules in the 200-350 nm region of the spectrum and choosing an appropriately designed plasmonic substrate that exhibits a resonance coincident with the excitation wavelength and absorption lines of biomolecules.

### BRIEF DESCRIPTION OF THE DRAWINGS

FIG. 1 is a schematic description of the sensing principle.

FIG. 2 is a schematic description of the sensor surface of FIG. 1 showing adsorbed species.

FIGS. 3A, 3B, 3C, 3D, and 3E is a collection of example absorbance spectra of nucleic acid bases, amino acid residues, proteins, nucleic acids, and small drug molecules in the UV range, wherein FIG. 3B is associated to FIG. 3C.

FIG. 4 is a schematic view of a sinusoidal profile surface relief grating substrate of FIG. 1 with incident light.

FIG. 5 is a detailed view of the sensor surface with layer structure FIG. 4.

FIG. 6 is a schematic description of the optical setup to measure the reflected light from the plasmonic surface under several focusing conditions.

FIG. 7 is experimentally measured reflectance from a plasmonic surface of FIG. 11 as a function of wavelength at various angles.

FIG. 8 is a schematic view of a rectangular profile surface relief grating substrate with incident light.

FIG. 9 is a measurement result showing the effect of 1 μM BSA protein adsorption on the plasmonic resonance at near infrared wavelengths.

FIG. 10 is a measurement result showing the effect of 0.0015 μM BSA protein adsorption on the plasmonic resonance at 230 nm wavelength.

FIG. 11 is a measurement result showing the effect of 0.0000007 μM BSA protein adsorption on the plasmonic resonance at 230 nm wavelength.

FIG. 12 is a schematic description of the optical setup to image the reflected light from the plasmonic surface with collimated illumination.

FIG. 13 is a schematic description of the plasmonic surface integrated with a microfluidic chamber and UV transparent window.

FIG. 14 is a schematic description of the plasmonic sensor integrated with a photodiode for measurement of transmitted light.

### DETAILED DESCRIPTION OF THE INVENTION

All illustrations of the drawings are for the purpose of describing selected versions of the present invention and are not intended to limit the scope of the present invention.

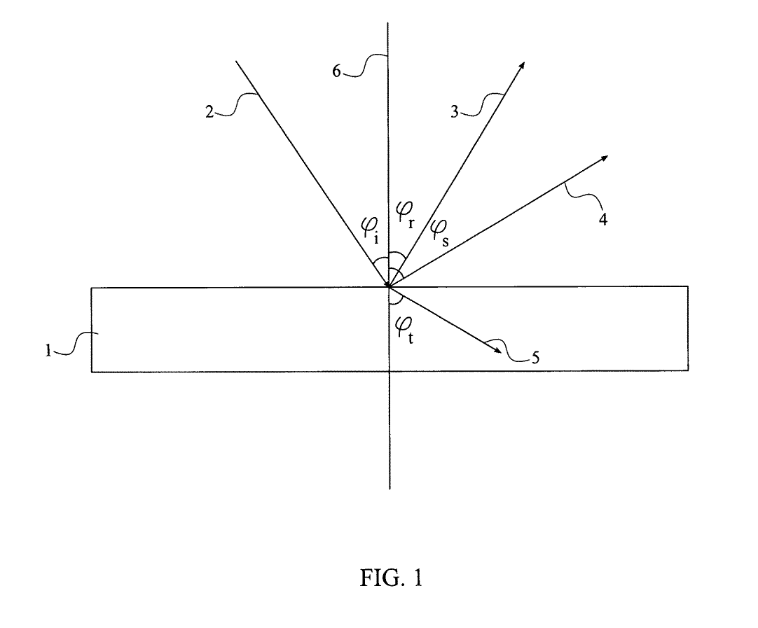

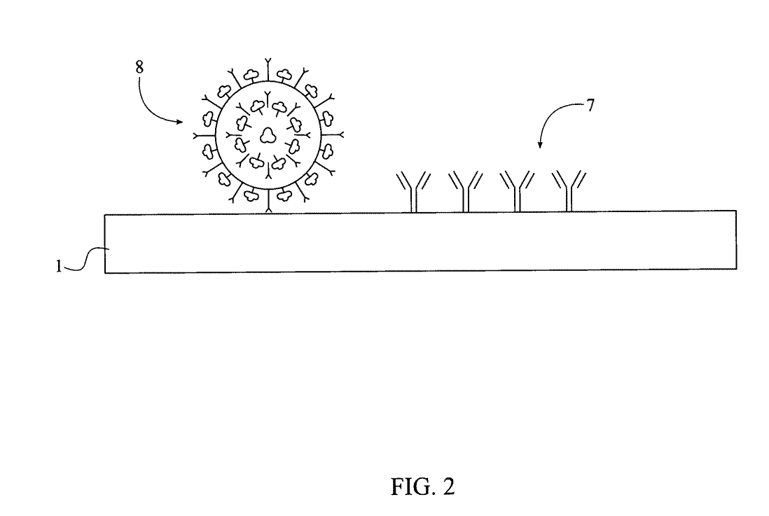

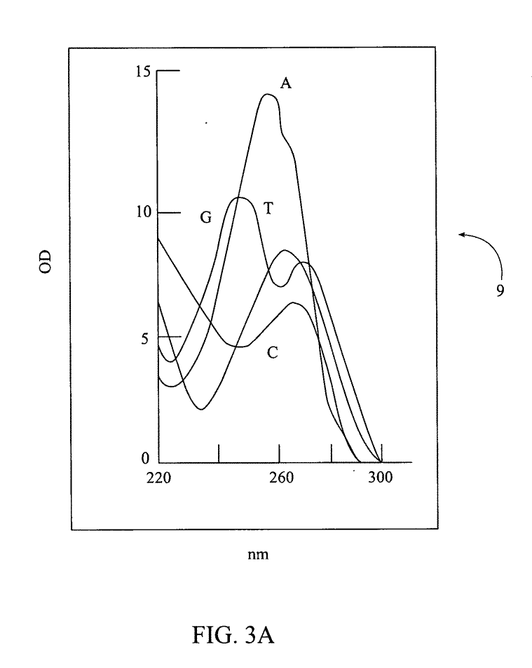

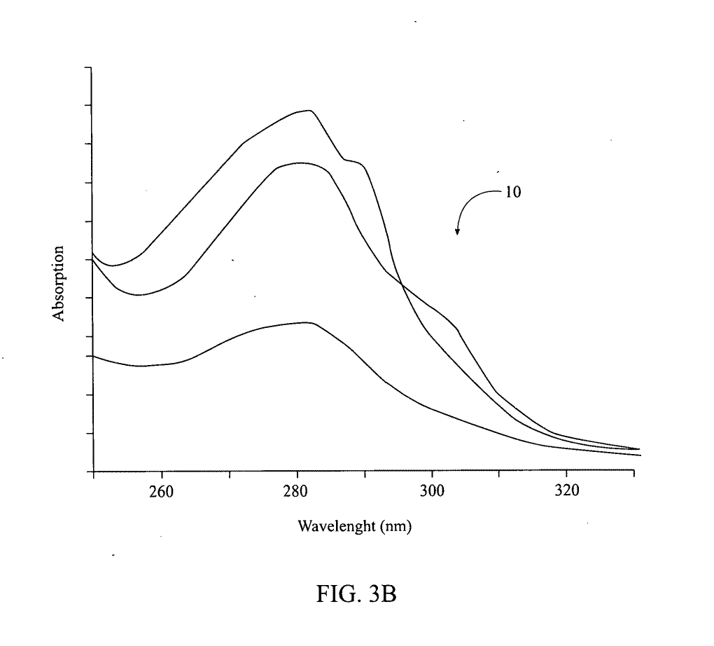

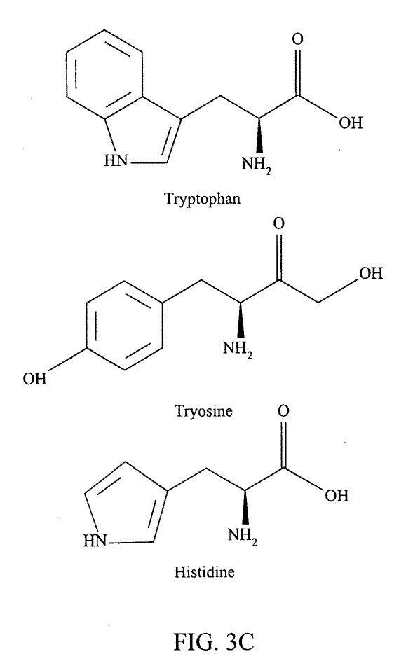

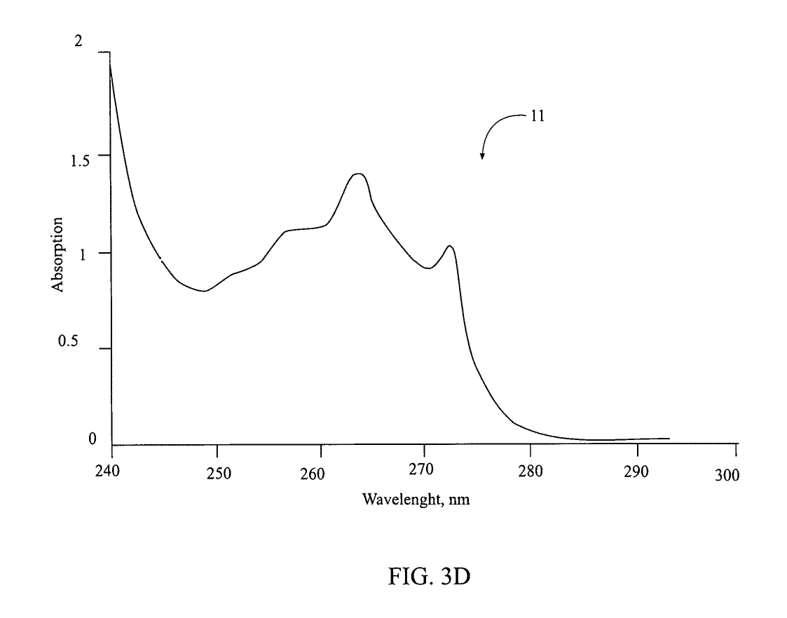

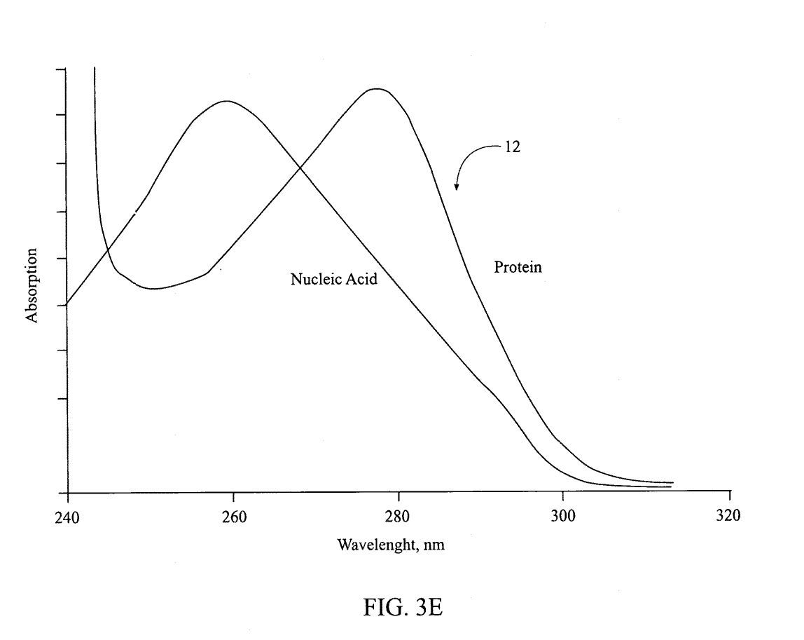

Referring now to the present invention in more detail, in FIG. 1, there is shown the UV Plasmonic Substrate (UVPS) 1, illuminated by an incoming light wave referred to as the excitation 2 (i.e. an incident UV light wave). A resultant portion could reflect off the UVPS 1 resulting in the reflection 3, get scattered resulting in the scattering 4, or get transmitted resulting in the transmission 5. The angle of incidence (AOI), or angle of scattering are the angles the excitation 2 or the scattering 4 makes with the surface normal 6 of the UVPS 1 (i.e. a receiving surface). In more detail, in FIG. 2, biomolecules 7 or biomolecular structures 8 (i.e. biomolecular analyte), are shown adsorbed onto the UVPS 1. Biomolecules 7 or biomolecular structures 8 may or may not be contained in a testing solution, which is place in contact with the receiving surface. In FIG. 3, native absorbance spectra of nucleic acid bases 9, native absorbance spectra of three amino acids 10, example drug molecules 11, and comparative absorption spectra of nucleic acids and proteins 12 are shown to exhibit strong absorption features in the ultraviolet region of the spectrum (UV). In one instance, the incident UV light wave is within a wideband spectrum ranging between 200 nm wavelength to 350 nm wavelength and is spatially configured to be an approximate line source. In another instance, the incident UV light wave is within a narrowband spectrum ranging at a fixed wavelength and is spatially configured to be an approximate point source.

In one embodiment of the UVPS 1, a surface relief grating 13 has a flat profile, which is made out of aluminum or is coated with an aluminum layer with a thickness ranging between 50 nm to 100 nm.

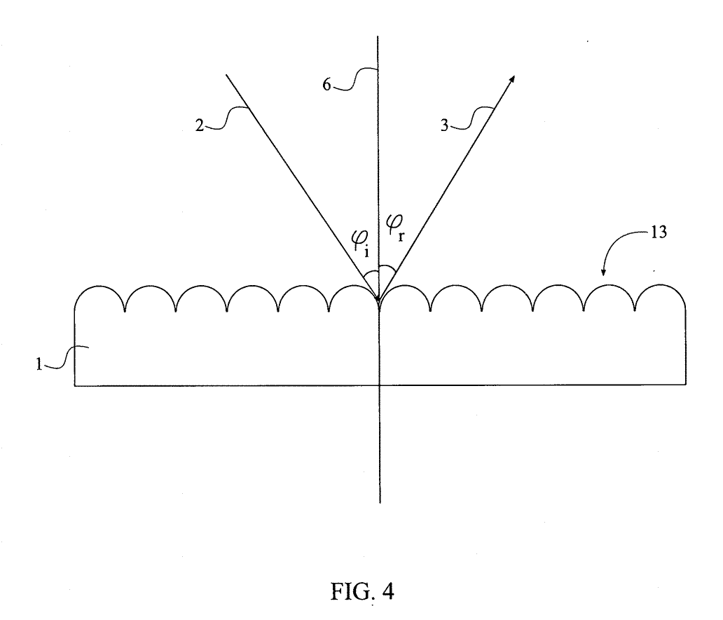

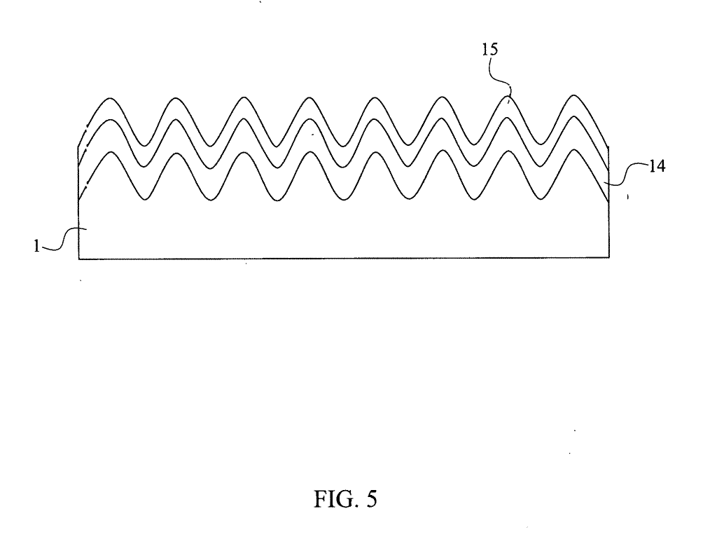

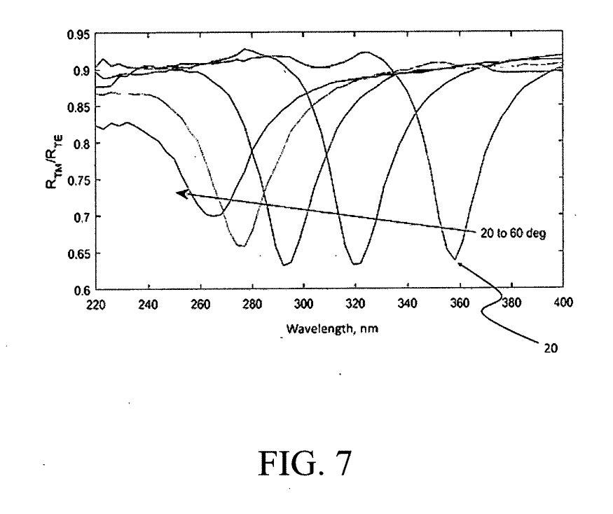

In another embodiment of the UVPS 1 shown in FIG. 4, a surface relief grating 13 with a sinusoidal-profile is embossed on to the UVPS 1, which is also made out of aluminum. In FIG. 5, a more detailed cross section of the UVPS 1 is given, where the surface relief grating 13 has two coating layers: a 1-3 nm thick oxide dielectric layer 14 (e.g. Al₂O₃, ZnO, or SiO₂) and a 0.3-2 nm thick adhesion layer 15 (e.g. gold). In more detail, still referring to the present invention shown in FIG. 5, the period and corrugation depth of the surface relief grating 13 is chosen such that the UVPS 1 exhibits optical resonances 20, that overlap in wavelength with the absorption of the biomolecules 7 and biomolecular structures 8, in the UV range for AOI in the angular range 0 to 89 degrees, as shown in FIG. 7. The sinusoidal profile is preferably shaped with a period ranging between 50 nm and 500 nm and is preferably shaped with a corrugation depth ranging between 10 nm and 50 nm

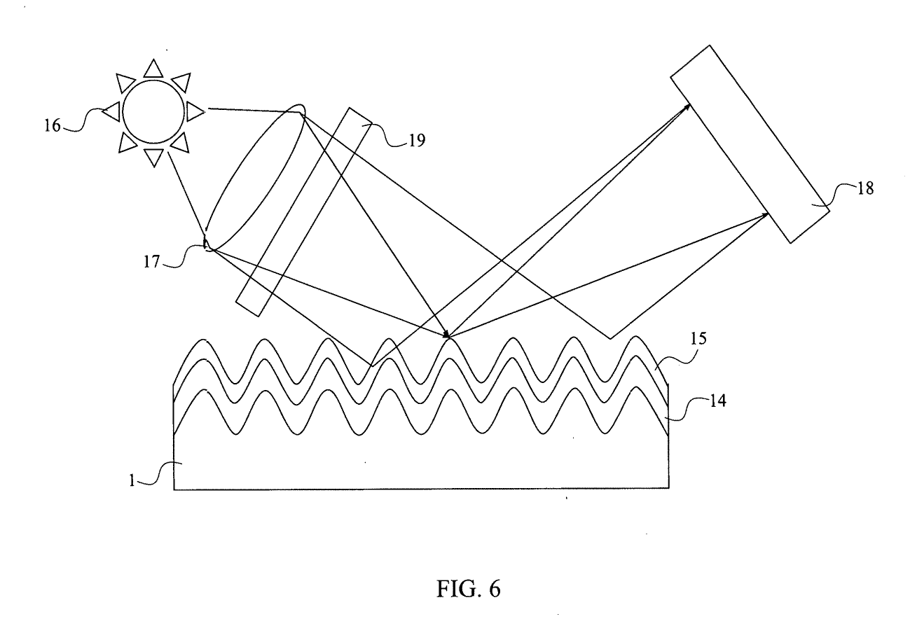

Now referring to FIG. 6, the method of measurement of the optical resonances 20 involves an optical setup with a point UV light source 16, collimated or focused by a UV transparent lens 17, where the light subsequently passes through a UV polarizer 19 and is incident on the UVPS 1, reflects off the surface of the UVPS 1 and is incident on a one-or-two-dimensional photodetector array 18.

In more detail, still referring to the present invention shown in FIG. 1 and the optical setup shown in FIG. 6, the angle or wavelength dependent optical resonances 20 are measured by the photodetector array 18 as a function of angle or wavelength and shift and quality factor of the resonances 20 are calculated. Due to the overlapping absorption spectra 9, 10, 11, 12 of the biomolecules 7 and biomolecular structures 8, the presence or absence of the biomolecules 7 and biomolecular structures 8 can be identified with sensitivity surpassing that of conventional SPR sensors that do not operate at UV wavelengths (200-350 nm).

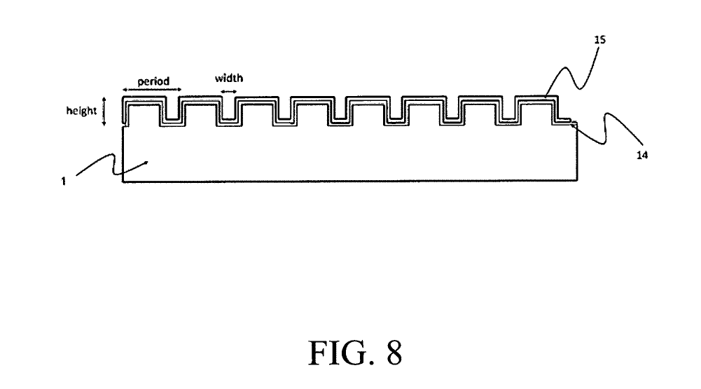

Referring now to the UVPS 1 shown in FIG. 8, the surface relief grating 13 may have a rectangular profile and is made of Aluminum, while similarly having the chemical protection dielectric layer 14 and the adhesion layer 15 as the sinusoidal profile. The period, width, and height of the rectangular profile are optimized to exhibit optical resonances 20 in the UV part of the spectrum (200-350 nm) for AOI in the angular range 0 to 89 degrees. The rectangular profile is preferably shaped with a duty cycle ranging between 1% and 50% and is preferably shaped with a corrugation depth ranging between 10 nm and 100 nm

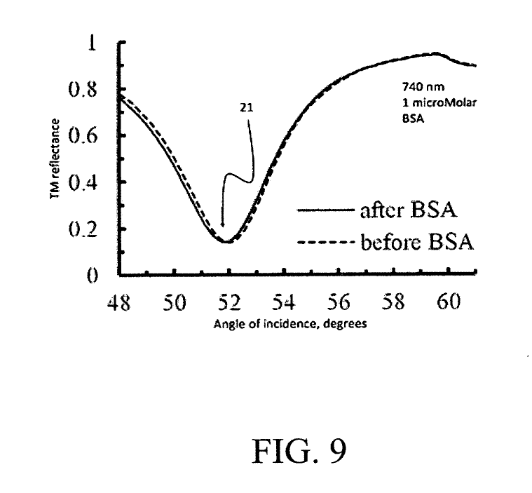

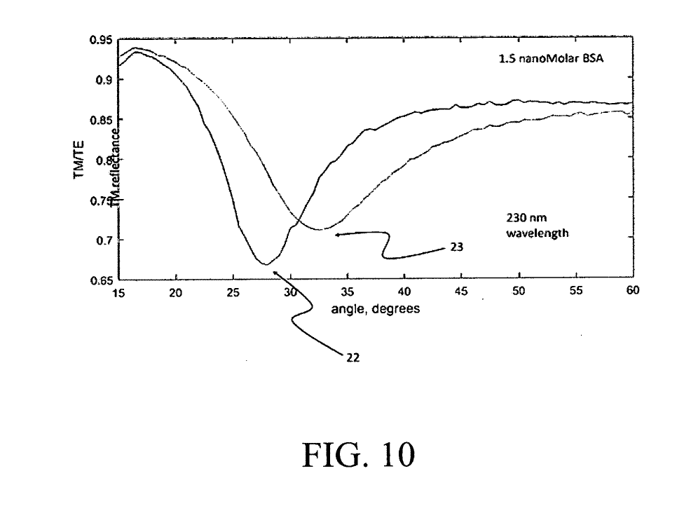

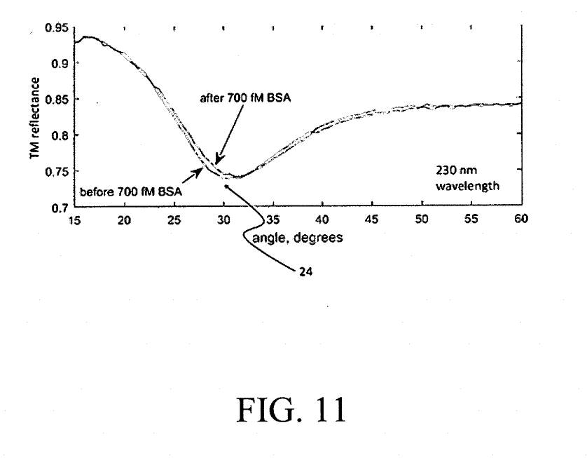

The advantages of the present invention include, without limitation, a highly enhanced sensitivity to the presence of the biomolecules 7 and biomolecular structures 8 compared to plasmon resonance sensors without overlapping molecular and plasmonic resonances. Referring to FIGS. 9 and 10, the shift 21 of the optical resonances 20 measured with the excitation 2 having a 740 nm wavelength for 1 microMolar (μM) Bovine Serum Albumin (BSA) protein exposure is much smaller as compared to the shift 23 of the optical resonances 20 measured with the excitation 2 having a 230 nm wavelength for 1.5 nanoMolar (nM) BSA protein exposure. Referring to FIG. 11, the sensing method produces a measurable shift 24, even when the BSA protein exposure is in the 700 femtoMolar (fM) range.

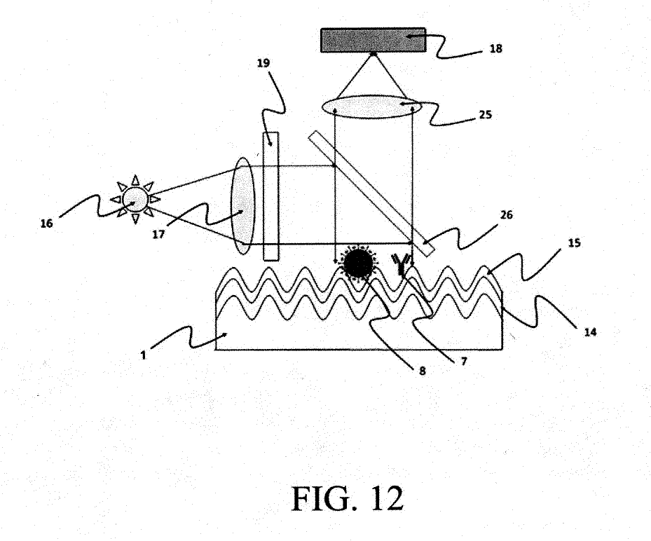

In another embodiment of the invention, referring to FIG. 12, the UV light source 16 is collimated by the UV transparent lens 17, is polarized in the transverse magnetic polarization by the polarizer 19, and is partially reflected on a 50/50 beam splitter 26, subsequently reflects from the UVPS 1, passes through the beam splitter 26, and is imaged by using a UV transparent imaging lens 25 and the photodetector array 18. When the excitation wavelength of the UV light source 16, matches the optical resonance of the UVPS 1 under normal incidence, and also matches the absorption 9, 10, 11, 12 of the biomolecules 7 and biomolecular structures 8, enhanced contrast imaging of the adsorption of the biomolecules 7 and the biomolecular structures 8 is achieved.

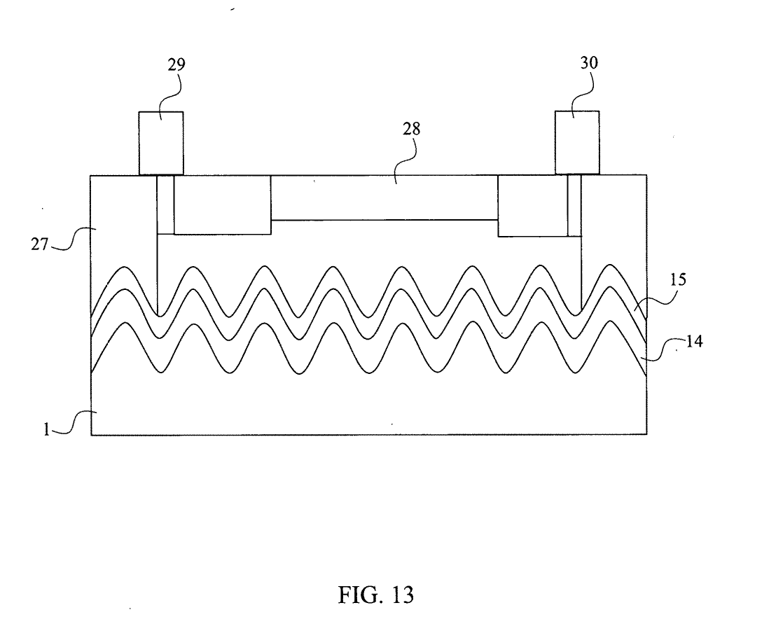

Referring now to FIG. 13, the UVPS 1 is integrated with a microfluidic chamber 31, that comprises a support 27, fluid inlet 29, fluid outlet 30, and a UV transparent window 28, which are used to draw the testing solution onto the receiving surface of the UVPS 1. The embodiments of the invention described in FIGS. 6 and 12 can be used as the fluidic integrated version of the UVPS 1.

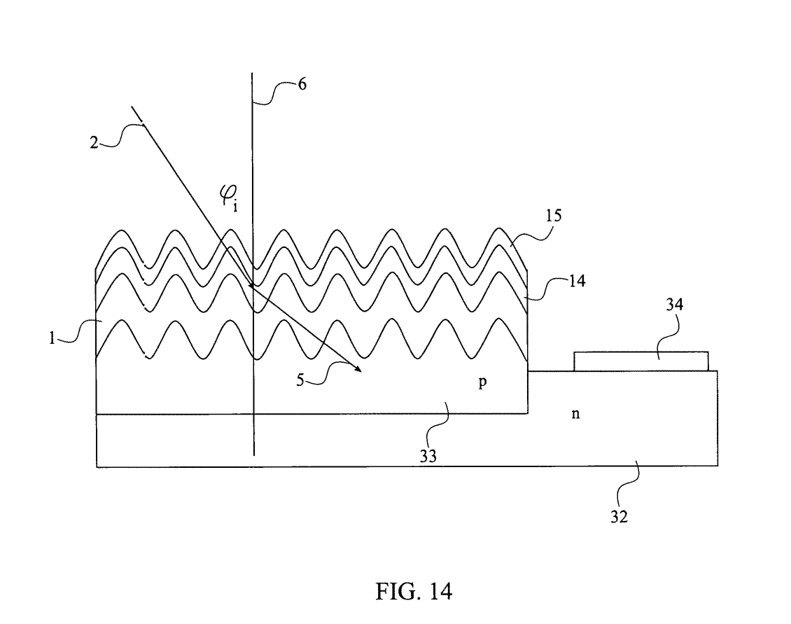

In another embodiment of the invention, referring to FIG. 14, the UVPS 1, is integrated onto a UV sensitive P-N junction photodiode, made of a semiconductor like silicon or gallium nitride (GaN), comprising a P-type region 33 and an N-type region 32 and an electrical body contact 34. The aluminum region of the UVPS 1 is chosen to be thin enough (5-50 nm thickness) so as to allow transmission of the resonantly coupled light into the surface plasmon mode of the UVPS 1, which also allows partial transmission of the UV light coupled to the surface plasmon polariton. The aluminum region of the UVPS 1 serves as an electrical contact to the P-type region 33. The transmitted light 5 is measured to discriminate between the presence or absence of the biomolecules.

In broad embodiment, the present invention is an optical method for the enhancement of sensitivity in biomolecular sensing by choosing a plasmonic substrate that exhibits resonances in the UV part of the spectrum, coinciding with native UV absorption spectra of biomolecules.

In a specific method of selecting a plurality of optical constants and an absorbed biomolecular layer thickness, the present invention is provided with standard reflectance data from the plasmonic substrate with absorbed biomolecules and is provided with an algorithm configured to calculate a theoretical reflectance for a given optical configuration for a plurality of optical constants and adsorbed biomolecular layer thickness. Moreover, the reflectance data includes a plurality of angle of incidence ranging between 0 degrees to 88 degrees and is a wavelength ranging between 200 nm and 350 nm. Thus, the plurality of optical constants and absorbed biomolecular layer thickness is selected in accordance to a best fit of the standard reflectance data by minimizing a square mean error between the standard reflectance data and the set of calculation results.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

Although the invention has been explained in relation to its preferred embodiment, it is to be understood that many other possible modifications and variations can be made without departing from the spirit and scope of the invention as hereinafter claimed.

What is claimed is:

1. A method for determining the presence or absence of adsorbed biomolecules or biomolecular structures on a surface, the method comprises the steps of:
(A) providing a plasmonic substrate, wherein a receiving surface of the plasmonic substrate exhibits plasmonic resonances at an ultraviolet (UV) wavelength range;
(B) placing a test solution in contact with the receiving surface;
(C) exciting the plasmonic substrate with an incident UV light wave with transverse magnetic polarization as the incident UV light wave illuminates the testing solution and the receiving surface;
(D) measuring a surface plasmon resonance (SPR) profile for a resultant portion of the incident UV light wave, wherein the resultant portion is either reflected, scattered, or transmitted from the incident UV light wave; and
(E) detecting a biomolecular analyte within the test solution, if the SPR profile for the resultant portion indicates overlapping molecular and plasmonic absorption spectra.

2. The method for determining the presence or absence of adsorbed biomolecules or biomolecular structures on a surface, the method as claimed in claim 1, wherein the UV wavelength range is between 220 nanometers (nm) to 350 nm.

3. The method for determining the presence or absence of adsorbed biomolecules or biomolecular structures on a surface, the method as claimed in claim 1, wherein the receiving surface is a flat surface.

4. The method for determining the presence or absence of adsorbed biomolecules or biomolecular structures on a surface, the method as claimed in claim 3, wherein the flat surface is made of aluminum or is coated with an aluminum layer with a thickness ranging between 50 nm to 100 nm.

5. The method for determining the presence or absence of adsorbed biomolecules or biomolecular structures on a surface, the method as claimed in claim 1, wherein the receiving surface is embossed with a sinusoidal surface relief grating.

6. The method for determining the presence or absence of adsorbed biomolecules or biomolecular structures on a surface, the method as claimed in claim 5, wherein the sinusoidal surface relief grating is shaped with a period ranging between 50 nm and 500 nm and is shaped with a corrugation depth ranging between 10 nm and 50 nm.

7. The method for determining the presence or absence of adsorbed biomolecules or biomolecular structures on a surface, the method as claimed in claim 5, wherein the sinusoidal surface relief grating is coated with a chemical protection layer with a thickness ranging between 1 nm to 3 nm, and wherein the chemical protection layer is an oxide layer.

8. The method for determining the presence or absence of adsorbed biomolecules or biomolecular structures on a surface, the method as claimed in claim 7, wherein the chemical protection layer is coated with an adhesion layer with a thickness ranging between 0.3 nm to 2 nm, and wherein the adhesion layer is made of gold.

9. The method for determining the presence or absence of adsorbed biomolecules or biomolecular structures on a surface, the method as claimed in claim 1, wherein the receiving surface is embossed with a rectangularly-profiled surface relief grating.

10. The method for determining the presence or absence of adsorbed biomolecules or biomolecular structures on a surface, the method as claimed in claim 10, wherein the rectangularly-profiled surface relief grating is shaped with a duty cycle ranging between 1% and 50% and is shaped with a corrugation depth ranging between 10 nm and 100 nm.

11. The method for determining the presence or absence of adsorbed biomolecules or biomolecular structures on a surface, the method as claimed in claim 10, wherein the rectangularly-profiled surface relief grating is coated with a chemical protection layer with a thickness ranging between 1 nm to 3 nm, and wherein the chemical protection layer is an oxide layer.

12. The method for determining the presence or absence of adsorbed biomolecules or biomolecular structures on a surface, the method as claimed in claim 11, wherein the chemical protection layer is coated with an adhesion layer with a thickness ranging between 0.3 nm to 2 nm, and wherein the adhesion layer is made of gold.

13. The method for determining the presence or absence of adsorbed biomolecules or biomolecular structures on a surface, the method as claimed in claim 1, wherein the plasmonic substrate is a UV sensitive photodiode or a UV sensitive photodiode array, and wherein the plasmonic substrate is made of silicon or gallium nitride (GaN).

14. The method for determining the presence or absence of adsorbed biomolecules or biomolecular structures on a surface, the method as claimed in claim 13, wherein the receiving surface is coated with a thin aluminum grating layer with a thickness ranging between 10 nm to 50 nm, and wherein the receiving surface is configured for partial transmission of the incident UV light wave.

15. The method for determining the presence or absence of adsorbed biomolecules or biomolecular structures on a surface, the method as claimed in claim 1, wherein a microfluidic chamber is operatively integrated into the plasmonic substrate, wherein the microfluidic chamber is used to draw the test solution onto the receiving surface.

16. The method for determining the presence or absence of adsorbed biomolecules or biomolecular structures on a surface, the method as claimed in claim 1 comprises the steps of:
providing the resultant portion as a reflectance of the plasmonic surface; and
measuring the SPR profile of the resultant portion during step (D) as a function of incidence angle and wavelength;

17. The method for determining the presence or absence of adsorbed biomolecules or biomolecular structures on a surface, the method as claimed in claim 1, wherein the incident UV light wave is within a wideband spectrum ranging between 200 nm wavelength to 350 nm wavelength and is spatially configured to be an approximate line source.

18. The method for determining the presence or absence of adsorbed biomolecules or biomolecular structures on a surface, the method as claimed in claim 1, wherein the incident UV light wave is within a narrowband spectrum ranging at a fixed wavelength and is spatially configured to be an approximate point source.

19. The method for determining the presence or absence of adsorbed biomolecules or biomolecular structures on a surface, the method as claimed in claim 1, wherein the incident UV light wave is conditioned into a collimated or focused beam by traversing through at least one UV transparent lens.

20. The method for determining the presence or absence of adsorbed biomolecules or biomolecular structures on a surface, the method as claimed in claim 1, wherein a polarization of the incident UV light wave is conditioned by traversing through a UV polarizer.

21. The method for determining the presence or absence of adsorbed biomolecules or biomolecular structures on a surface, the method as claimed in claim 1 comprises the steps of:
providing an optical detector; and
measuring the SPR profile of the resultant portion during step (D) with the optical detector.

22. The method for determining the presence or absence of adsorbed biomolecules or biomolecular structures on a surface, the method as claimed in claim 21, wherein the optical detector is a one-dimensional linear sensor and is configured to be sensitive to the UV region of the electromagnetic (EM) spectrum.

23. The method for determining the presence or absence of adsorbed biomolecules or biomolecular structures on a surface, the method as claimed in claim 21, wherein the optical detector is a two-dimensional linear sensor and is configured to be sensitive to the UV region of the EM spectrum.

24. The method for determining the presence or absence of adsorbed biomolecules or biomolecular structures on a surface, the method as claimed in claim 21, wherein the optical detector is a spectrometric sensor and is configured to be sensitive to the optical region and the UV region of the EM spectrum.

25. The method for determining the presence or absence of adsorbed biomolecules or biomolecular structures on a surface, the method as claimed in claim 1 comprises the steps of:
providing a beam splitter and an optical detector, wherein the beam splitter is positioned along an optical path traversed by the incident UV light wave and the resultant portion;
optically splitting the incident UV light wave into a first intermediate portion and a second intermediate portion;
routing the first intermediate portion from the beam splitter, then to the plasmonic substrate, back to the beam splitter, and then to the optical detector; and
routing the second intermediate portion from the beam splitter and then to the optical detector.

26. The method for determining the presence or absence of adsorbed biomolecules or biomolecular structures on a surface, the method as claimed in claim 1 comprises the steps of:
providing a plurality of UV transparent lenses and a UV sensitive imaging photodetector array; and
measuring the SPR profile of the resultant portion during step (D) with the UV sensitive imaging photodetector array by optically guiding the incident UV light wave and the resultant portion through the plurality of UV transparent lenses.

27. The method for determining the presence or absence of adsorbed biomolecules or biomolecular structures on a surface, the method as claimed in claim 1 comprises the steps of:
providing standard reflectance data from the plasmonic substrate with absorbed biomolecules, wherein the reflectance data includes a plurality of angle of incidence ranging between 0 degrees to 88 degrees and is a wavelength ranging between 200 nm and 350 nm;
providing an algorithm configured to calculate a theoretical reflectance for a given optical configuration for a plurality of optical constants and adsorbed biomolecular layer thickness;
executing the algorithm in order to generate a set of calculation results; and
selecting the plurality of optical constants and absorbed biomolecular layer thickness in accordance to a best fit of the standard reflectance data by minimizing a square mean error between the standard reflectance data and the set of calculation results.
